(19) United States
(12) Patent Application Publication
(10) Pub. No.: US 2025/0127816 A1
(43) Pub. Date: Apr. 24, 2025

(54) SYSTEMS AND METHODS FOR GROWING MITOCHONDRIA AND COATING THEREOF

(71) Applicant: MITRIX BIO, INC., Pleasanton, CA (US)

(72) Inventor: Thomas B. Benson, Los Altos, CA (US)

(21) Appl. No.: 19/005,419

(22) Filed: Dec. 30, 2024

Related U.S. Application Data
(63) Continuation of application No. PCT/US2023/027040, filed on Jul. 6, 2023.
(60) Provisional application No. 63/368,033, filed on Jul. 8, 2022.
(60) Provisional application No. 63/368,152, filed on Jul. 11, 2022.

(51) Int. Cl. A61K 35/19; C12N 5/00; C12N 5/078

(52) U.S. Cl. CPC A61K 35/19; C12N 5/0006; C12N 5/0644

(57) ABSTRACT

A method of growing isolated mitochondria includes selecting a source of mitochondria and stem cells, extracting the stem cells and the mitochondria from the source and segregating the stem cells and the mitochondria into first and second pools respectively, incubating/transferring the extracted mitochondria into the extracted stem cells to produce packed stem cells, expanding the packed stem cells in a bioreactor, adjusting conditions of an environment of the bioreactor to favor growth of the mitochondria of the packed stem cells, converting the packed stem cells into megakaryocytes, isolating the mitochondria from the megakaryocytes, and applying a coating to the mitochondria following their isolation from the megakaryocytes.

### INCORPORATION BY REFERENCE TO ANY PRIORITY APPLICATIONS

This application is a continuation of PCT Application Serial No. PCT/US2023/027040, filed Jul. 6, 2023, which claims priority to U.S. Provisional Application Ser. Nos. 63/368,033, filed Jul. 8, 2022 and 63/368152, filed Jul. 11, 2022. Each of the aforementioned applications is incorporated herein by reference in its entirety.

### BACKGROUND

The presently disclosed and claimed inventions relate generally to a systems and methods for growing mitochondria and coating thereof. More particularly, systems and methods for obtaining bioreactor-grown, coated mitochondria for treatment of various pathologies or for maintaining/increasing cellular energetics aimed at promoting longevity.

Researchers have been testing exogenous mitochondrial transplants, but this has been confined largely to rare pediatric diseases and surgery, not the larger world of adult diseases and longevity, because of scarce supplies of donor mitochondria. Just as organ transplants, such as liver or kidney, are severely limited by availability of donor organs, so too mitochondrial “organelle transplants” are limited by scarce supplies of donor mitochondria. Mitochondria are “the powerplants of the cell”—tiny organelles that generate the energy cells need to replicate and function. Research has shown mitochondria are highly mobile, transferring from cell to cell directly and via the bloodstream, and that dysfunction of mitochondria due to injury, age, or mutation can cause disease. Accordingly, scarcity in donor mitochondria that are available for treatment of adult diseases has long stunted the field for mitochondrial-based therapeutics, and thus has been one of the main difficulties in expanding such therapeutics beyond the presently small confines. Therefore, there has been a long-felt need to develop systems and methods to grow mitochondria and increase the efficacy of cellular uptake of such mitochondria.

### SUMMARY

The methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

In some embodiments, a method of growing isolated mitochondria is provided, the method including selecting a source including mitochondria and stem cells; extracting the stem cells and the mitochondria from the source and segregating the stem cells and the mitochondria into first and second pools respectively; incubating/transferring the extracted mitochondria into the extracted stem cells to produce packed stem cells; expanding the packed stem cells in a bioreactor; adjusting conditions of an environment of the bioreactor to favor growth of the mitochondria of the packed stem cells; converting the packed stem cells into megakaryocytes; isolating the mitochondria from the megakaryocytes; and applying a coating to the mitochondria following isolation thereof from the megakaryocytes.

In some embodiments, the source is placenta tissue including the mitochondria and the stem cells. In some embodiments, the source is bone marrow including the mitochondria and the stem cells. In some embodiments, the source is adipose tissue including the mitochondria and the stem cells. In some embodiments, the source is peripheral blood including platelet-derived extracellular vesicles (PEVs). In some embodiments, the PEVs include the mitochondria.

In some embodiments, the coating includes asialoorosomucoid (AsOR). In some embodiments, the coating further includes a poly-L-lysine. In some embodiments, the coating further includes listeriolysin O (LLO).

In some embodiments, the method further includes storing the coated mitochondria. In some embodiments, the storing step includes suspending the mitochondria in a cryoprotectant. In some embodiments, the cryoprotectant includes trehalose. In some embodiments, the cryoprotectant includes phosphate buffered saline (PBS). In some embodiments, wherein the coating is applied at a ratio of 1 μg of the coating for 128 μg of the bioreactor-grown mitochondria. In some embodiments, the coating is applied at a ratio that is at least double of 1 μgof the coating for 128 μg of the bioreactor-grown mitochondria.

In some embodiments, the method further includes administering a therapeutic amount of the coated mitochondria to a subject.

In some embodiments, the method further includes obtaining blood from one or more donors; adding an anticoagulant and a buffer to the blood to form a mix; separating the mix into supernatant and platelet rich plasma (PRP); collecting the PRP; stimulating the collected PRP, thereby expelling extracellular vesicles from platelets in the PRP; and collecting the extracellular vesicles as the PEVs. In some embodiments, the collected PRP is stimulated with immune complexes in presence of Ca²⁺. In some embodiments, the immune complexes include heat-aggregated IgG. In some embodiments, a concentration of the heat-aggregated IgG is 0.1 mg/mL to 2.5 mg/mL, and wherein concentration of the Ca²⁺ is 1 mM to 25 mM. In some embodiments, the anticoagulant is anticoagulant citrate dextrose (ACD). In some embodiments, the buffer is Tyrode's buffer at pH 6 to pH 7.

In some embodiments, a method of growing isolated mitochondria is provided, the method including selecting a source including mitochondria and stem cells; extracting the stem cells and the mitochondria from the source and segregating the stem cells and the mitochondria into first and second pools respectively; incubating/transferring the extracted mitochondria into the extracted stem cells to produce packed stem cells; expanding the packed stem cells in a bioreactor; adjusting conditions of an environment of the bioreactor to favor growth of the mitochondria of the packed stem cells; converting the packed stem cells into megakaryocytes; and isolating the mitochondria from the megakaryocytes. In some embodiments, the method further includes applying a coating to the mitochondria following isolation thereof from the megakaryocytes.

In some embodiments, the source is placenta tissue including the mitochondria and the stem cells. In some embodiments, the source is bone marrow including the mitochondria and the stem cells. In some embodiments, the source is adipose tissue including the mitochondria and the stem cells. In some embodiments, the source is peripheral blood including platelet-derived extracellular vesicles (PEVs). In some embodiments, the PEVs include the mitochondria.

In some embodiments, the coating includes asialoorosomucoid (AsOR). In some embodiments, the coating further includes a poly-L-lysine. In some embodiments, the coating further includes listeriolysin O (LLO).

In some embodiments, the method further includes storing the coated mitochondria. In some embodiments, the storing step includes suspending the mitochondria in a cryoprotectant. In some embodiments, the cryoprotectant includes trehalose. In some embodiments, the cryoprotectant includes phosphate buffered saline (PBS). In some embodiments, the coating is applied at a ratio of 1 μgof the coating for 128 μg of the bioreactor-grown mitochondria. In some embodiments, the coating is applied at a ratio that is at least double of 1 μgof the coating for 128 μg of the bioreactor-grown mitochondria.

In some embodiments, the method further includes administering a therapeutic amount of the coated mitochondria to a subject.

In some embodiments, the method further includes obtaining blood from one or more donors; adding an anticoagulant and a buffer to the blood to form a mix; separating the mix into supernatant and platelet rich plasma (PRP); collecting the PRP; stimulating the collected PRP, thereby expelling extracellular vesicles from platelets in the PRP; and collecting the extracellular vesicles as the PEVs. In some embodiments, the collected PRP is stimulated with immune complexes in presence of Ca²⁺. In some embodiments, the immune complexes include heat-aggregated IgG. In some embodiments, a concentration of the heat-aggregated IgG is 0.1 mg/mL to 2.5 mg/mL, and wherein concentration of the Ca2+ is 1 mM to 25 mM. In some embodiments, the anticoagulant is anticoagulant citrate dextrose (ACD). In some embodiments, the buffer is Tyrode's buffer at pH 6 to pH 7.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a flow diagram illustrating a method of growing isolated mitochondria according to some embodiments.

FIG. 2 illustrates a method of growing large volumes of mitochondria and the coating thereof for therapeutic use according to some embodiments.

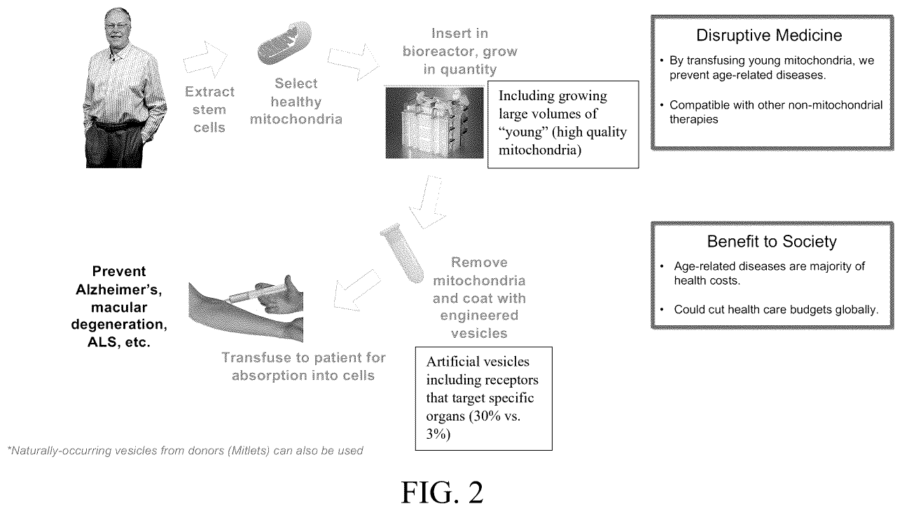

FIG. 3 illustrates a method of growing multiple types of transplantable mitochondrial species according to some embodiments.

FIG. 4 illustrates a method of growing multiple types of mitochondria and the coating thereof for transport and transfusion according to some embodiments.

FIG. 5 provides results of improved yields of mitochondria isolation from megakaryocytes according to some embodiments.

FIG. 6 shows results from a non-limiting example of basal cell respiration rescue via mitochondrial transfusion into cells according to some embodiments.

FIG. 7 shows flow cytometry results related to the coating of mitochondria according to a non-limiting example of some of the embodiments.

FIG. 8 shows flow cytometry results related to coating of mitochondria according to a non-limiting example of some of the embodiments.

FIG. 9 shows the basal respiration at 24 hours measured by the oxygen consumption rate between mitochondria coated under various formulations and mitochondria that are uncoated according to a non-limiting example.

FIG. 10 depicts the basal respiration of FIG. 9 with only 10 pg of mitochondria per cell shown.

FIG. 11 shows the maximal respiration at 24 hours measured by the by the oxygen consumption rate between mitochondria coated under various formulations and mitochondria that are uncoated according to a non-limiting example.

FIG. 12 depicts the maximal respiration of FIG. 11 with only 10 pg of mitochondria per cell shown.

FIG. 13 shows results of a Seahorse XF mitochondria stress test between DMSO vs ddC treatment at 24 hours according to a non-limiting example.

FIG. 14 shows results of a Seahorse XF mitochondria stress test between DMSO vs ddC treatment at 72 hours according to a non-limiting example.

FIG. 15 shows the basal respiration at 72 hours measured by the oxygen consumption rate between mitochondria coated under various formulations and mitochondria that are uncoated according to a non-limiting example.

FIG. 16 depicts the basal of FIG. 15 with only results for 10 pg of mitochondria per cell shown.

FIG. 17 shows the maximal respiration at 72 hours measured by the oxygen consumption rate between mitochondria coated under various formulations and mitochondria that are uncoated according to a non-limiting example.

FIG. 18 depicts the maximal respiration of FIG. 17 with only results for 10 pg of mitochondria per cell shown.

FIGS. 19A-19D show basal respiration, maximal respiration, spare capacity, and proton leak results, respectively, measured by the oxygen consumption rate of ddC-treated cells and mitochondria according to a non-limiting example.

FIGS. 20A and 20B show ATP Production and mitochondrial respiration respectively according to a non-limiting example.

FIGS. 21A-21E show basal respiration, maximal respiration, spare capacity, proton leak, and ATP production, respectively, according to a non-limiting example.

FIGS. 22A and 22B show flow cytometry data related to the coating of mitochondria according to some embodiments.

FIG. 23 show protein concentrations of mitochondria samples following storage at various temperatures according to some embodiments.

FIG. 24 show mitochondria counts across different storage conditions according to some embodiments.

FIGS. 25A, 25B, and 25C show mtDNA results across different storage conditions according to some embodiments.

FIG. 26 shows ATP levels in mitochondria across different storage conditions according to some embodiments.

FIG. 27 shows results from a mitochondrial respiration assay across different storage conditions according to some embodiments.

FIG. 28 shows flow cytometry data for various cryoprotectants across different storage conditions according to some embodiments.

FIG. 29 shows an ALS animal model that receives mitochondria transfusions according to a non-limiting example.

FIG. 30 shows the development of phenotypes in the ALS animal model similar to that of ALS in humans.

FIG. 31 shows the experimental design for mitochondria transfusion in the ALS animal model.

FIGS. 32A-32D show results of a variety assessments of the efficacy of mitochondrial transfusion in an animal model of ALS.

### DETAILED DESCRIPTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. The use of the term “containing” as well as other forms, such as “contain,” “contains,” and “contained,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, the singular forms “a,” “an” and “the” include plural referents unless the context clearly dictates otherwise.

As used herein, the term “mitochondria” includes membrane-bound entities featuring a unique bilayer membrane construction. This membrane construction includes an outer membrane that is relatively permeable to small molecules, and an inner membrane which is highly impermeable, folded into structures known as cristae. The space enclosed by the inner membrane is referred to as the mitochondrial matrix. The functional aspects of the mitochondria, particularly its role in cellular respiration and energy production includes complex series of biochemical reactions, including, but not limited to, the citric acid cycle (Krebs Cycle) and oxidative phosphorylation, whereby mitochondria convert nutrients into adenosine triphosphate (ATP), the primary energy currency of the cell. Furthermore, the term “mitochondria” includes contributions from these membrane-bound entities to various cellular functions beyond energy production. These roles include, but are not limited to, involvement in cellular differentiation, cell death, cell cycle and cell growth regulation, and maintenance of intracellular calcium levels, among others. Additionally, “mitochondria” is utilized to describe the organelle's unique genetic characteristic. Each mitochondrion contains its own DNA, known as mitochondrial DNA (mtDNA), which is independent of nuclear DNA and primarily inherited maternally. As used herein, the term mtDNA includes a small, circular double-stranded DNA molecule that resides within the mitochondria, separate from the nuclear genome found in the cell nucleus.

As used herein, the terms “mito,” “mitos,” and “mitlets” include naked mitochondria and bioreactor-grown mitochondria unless otherwise specified. Naked mitochondria refers to mitochondria that were not grown nor expanded in a bioreactor. In other words, naked mitochondria may be derived from sources following digestion treatment and fractionation thereof to obtain a small amount of the mitochondria. Naked mitochondria suffers several drawbacks, such as low yields, aged mtDNA (and therefore less resilient to damage accumulated from reactive oxidative species), and susceptibility to immune responses.

As used herein, the term “bioreactor” includes an apparatus or device constructed to support the growth and proliferation of biological entities under controlled and regulated environmental conditions. The bioreactor includes a containment chamber, which is constructed from biocompatible materials to minimize any negative interaction with the biological entities it houses. This chamber provides a secluded and controlled environment, effectively preventing contamination from external sources and promoting optimal growth conditions for the biological entities to grow or proliferate therein in the presence of appropriate media. The “bioreactor” may further include a series of sensors designed to continuously monitor and record critical parameters including, but not limited to, temperature, pH, oxygen and carbon dioxide concentrations, and nutrient and waste product levels. The feedback from these sensors is essential to inform adjustments in the bioreactor's internal environment, thereby maintaining optimal growth conditions. The bioreactor may further include a control system operatively connected to these sensors and to the containment chamber. This control system interprets data from the sensors, and in response, adjusts the environmental conditions within the containment chamber. The controlled manipulation of these parameters allows for the fine-tuning of the biological entity's environment, leading to enhanced growth and productivity. The “bioreactor” may be configured as an input/output system structured for the introduction of fresh media, necessary for providing nutrients, and the removal of spent media, crucial for the elimination of waste products. This system ensures a dynamic environment within the containment chamber, supporting the sustained viability and optimal productivity of the biological entities.

As used herein, the term “stem cells” includes unspecialized biological cells capable of self-renewal and differentiation into specialized cell types. Stem cells encompass both embryonic stem cells (ESCs) and adult stem cells. ESCs are derived from the inner cell mass of blastocysts, the early-stage embryos. They are pluripotent in nature, implying they possess the ability to differentiate into any cell type present in the adult body. Adult stem cells, also known as somatic or tissue-specific stem cells, are found scattered throughout adult tissues, such as bone marrow, blood, brain, and skin. These cells, typically multipotent, can differentiate into a limited number of cell types related to the tissue in which they reside. The term “stem cells” also includes induced pluripotent stem cells (iPSCs), which are adult cells that have been genetically reprogrammed to an embryonic stem cell-like state. This reprogramming enables iPSCs to differentiate into virtually any cell type, providing a potentially unlimited source of any human cell type.

As used herein, the term “iPSCs” include adult somatic cells that have been genetically reprogrammed to an embryonic stem cell-like state. This reprogramming imbues these cells with pluripotency, the ability to differentiate into virtually any cell type found in the body. iPSCs are generated by introducing specific transcription factors, such as Oct4, Sox2, Klf4, and c-Myc into adult somatic cells. This reprogramming process resets the cellular state, allowing the modified cells to proliferate indefinitely and to differentiate into a wide range of cell types upon appropriate stimulation.

The present invention provides a manner to overcome the scarcity of supplies of donor mitochondria available to treat adult diseases. In some embodiments described herein, the first step is to grow mitochondria in bioreactors. Next, those mitochondria are given a unique coating to protect against immune reactions along with receptors to target specific tissue types. These coated mitochondria are infused into the body, where they travel by the blood to desired tissues, and take up residence in cells.

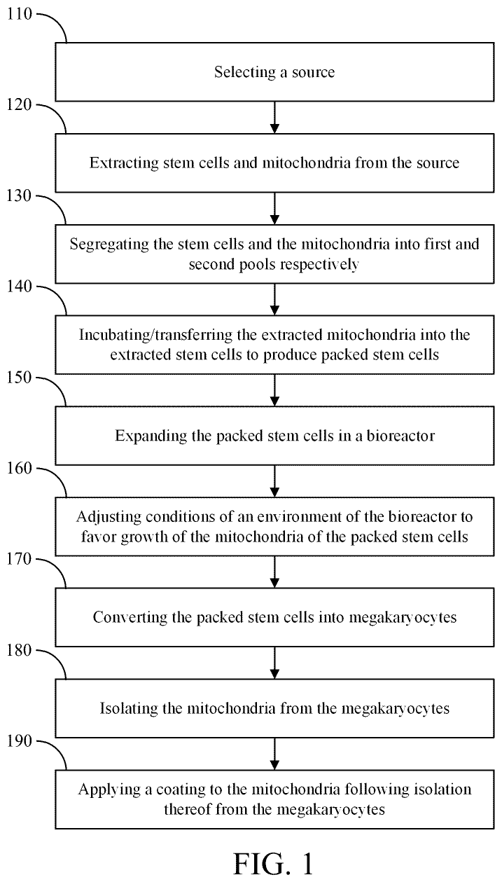

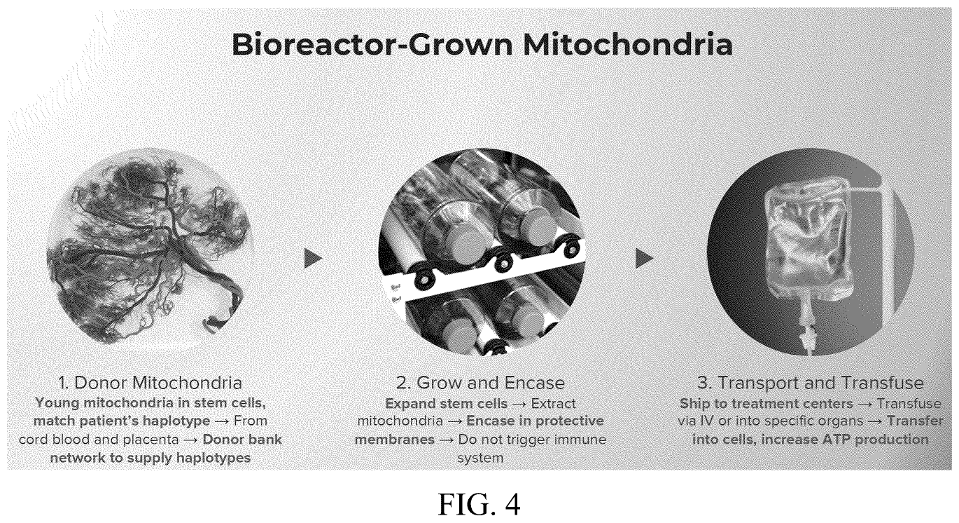

In some embodiments, a method 100 for growing mitochondria is provided as shown in FIG. 1, the method 100 including selecting a source in step 110. In some embodiments, the source includes mitochondria and stem cells. In some embodiments, the source includes naturally occurring vesicles from donor blood. In some embodiments, the source includes stem cells due to the typical younger age of the mitochondria, as evidenced by the lower reactive oxygen species damage accumulation in mtDNA in mitochondria from stem cells compared to mtDNA in mitochondria from older cells. In some embodiments, the stem cells match the subject's haplotype as shown in FIG. 4. In some embodiments, the stem cells include pluripotent stem cells. In some embodiments, the pluripotent stem cells include induced pluripotent stem cells. In some embodiments, the stem cells are obtained from cord blood. In some embodiments, the stem cells are obtained from the placenta. In some embodiments, the stem cells are obtained from dental pulp. In some embodiments, the stem cells are obtained from dermis. In some embodiments, the stem cells are obtained from amniotic fluid. In some embodiments, the stem cells are obtained from tumors. In some embodiments, the stem cells include hematopoietic stem cells obtained from the bone marrow. In some embodiments, the stem cells are obtained from adipose tissue. In some embodiments, the method 100 further includes extracting mitochondria from stem cells in step 120. In some embodiments, the method 100 includes selecting healthy mitochondria from the extracted mitochondria.

In some embodiments, the vesicles from donor blood are collected from platelets. In some embodiments, the obtaining of the mitochondria step includes extracting platelet-derived mitochondria-containing extracellular vesicles (PEVs). In some embodiments, the extraction step further includes: 1) obtaining blood from donors, 2) adding anticoagulant and a buffer to the blood to form a mix, 3) separating the mix into supernatant and platelet rich plasma (PRP), 4) collecting platelet rich plasma (PRP), 5) stimulating the collected platelets, and collecting the PEVs. Finding a source of mitochondria to for therapeutic use (e.g., administering mitochondria formulations) is a challenge. Just like any donated organ, mitochondria from young healthy donors are in short supply. Some diseases or injuries might be cured by autologous mitochondria-removed from a leg muscle in one's own body in a non-limiting example-however, for many other diseases, the “patients” have poor quality mitochondria due to age or mutation to mtDNA. For these patients, donated mitochondria are a preferred solution. In addition, freshly fully isolated mitochondria die quickly within minutes of isolation and may also provoke immune reactions when put naked into the bloodstream, reducing their effectiveness as a therapy. Therefore, it would be convenient to find an easy and readily available source of donation-ready mitochondria, which at the same time, are encased in some sort of coating, vesicle, or vehicle suspension (or any combination thereof) that protect them from the immune system. A platelet from human blood contains 4-5 mitochondria on average that are expelled in extracellular vesicles when platelets are activated. The platelet-derived mitochondria-containing extracellular vesicles are referred to PEVs herein. These PEVs are usually larger (>400 nM), and less well-known than other platelet extracts or lysates (30-100 nM), however other sizes may also apply. PEVs have been shown to donate their mitochondria to cells nearby, which can increase the respiratory activity of the cells that absorb them. PEVs have several advantages for fast commercialization: notably, they can be extracted from donated platelets that have “expired” and must be thrown away; they represent another good medically-valid use for platelets which otherwise might go to waste; they could be collected at most blood banks, who already have all the needed skilled personnel, clean handling practices, and equipment needed, and are already in close proximity to hospitals, thus making PEV product potentially available to world-wide use extremely soon. PEVs are a variety of platelet transfusion and therefore are more likely to be embraced and tested by medical professionals who are already familiar with blood transfusion therapies. Furthermore, PEVs can be prepared for localized transfusion into various internal anatomical regions to treat various clinical disorders using delivery devices already on the market.

Following the extraction step 120 of the method 100, stem cells and miscellaneous mitochondria are segregated and isolated into two different pools of material in step 130. In some embodiments, the first pool of material includes the stem cells. In some embodiments, the stem cells are isolated prior to the miscellaneous mitochondria. In some embodiments, the second pool includes miscellaneous mitochondria that is extracted from the remainder of the tissue from which stem cells were previously extracted. In some embodiments, the remainder of the tissue is grounded up prior to extracting the miscellaneous mitochondria. In some embodiments, the tissue includes umbilical cord blood. In some embodiments, the tissue includes an umbilical cord. In some embodiments, the tissue includes bone marrow. In some embodiments, the tissue includes adipose tissue. In some embodiments, the tissue includes any tissue that is associated with stem cells. In some embodiments, the tissue includes any stem-cell-including tissue.

In some embodiments, the method 100 further includes growing the mitochondria in a bioreactor. In some embodiments, the method 100 includes incubating/transferring the extracted mitochondria into the stem cells in step 140. In some embodiments, the method 100 includes expanding the packed stem cells in the bioreactor in step 150 as shown in FIGS. 1 and 4. In some embodiments, any extracted mitochondria not used in the stem cells can be used for therapeutics. In some embodiments, the step of growing the mitochondria in the bioreactor further includes expanding the stem cells packed with the mitochondria at the highest rate allowed by the bioreactor. In some embodiments, the method 100 further includes adjusting selection conditions in step 160 to favor high quality mitochondria. In some embodiments, the selection conditions include hypoxia, glucose starvation, use of a Treefrog process, or any combination thereof. In some embodiments, the method 100 further includes differentiating the remaining stem cells into megakaryocytes (MKs) in step 170, thereby increasing the numbers of mitochondria being produced a hundredfold, even a thousandfold, over traditional cell culture techniques.

As used herein, MKs are polyploid cells derived from hematopoietic stem cells that are found in the bone marrow. The role of MKs include generation of blood platelets that participate in localized clot formation to block hemorrhages. MKs that are grown in the bioreactor can come from not only hematopoietic stem cells found in the bone marrow. In some embodiments, MKs can be generated/induced from stem cells that are sourced from adipose tissue. In some embodiments, the adipose tissue includes subcutaneous adipose tissue. In some embodiments, the method further includes obtaining the source. In some embodiments, the source comprises adipose tissue, wherein the adipose tissue is obtained by any known technique in the art. In some embodiments, after obtaining the adipose tissue, the method further includes digesting the adipose tissue with a digestion agent. In some embodiments, the digestion agent is collagenase type II. In some embodiments, the method further includes centrifuging the digested adipose tissue to generate an adipose-derived mesenchymal stromal stem cell line (ASCL). In some embodiments, the method further includes treating the ASCL with MK lineage induction media to generate the MKs. In some embodiments, the MK lineage induction media includes: 2 mM L-glutamine; 100 U/mL penicillin-streptomycin solution; 0.5% bovine serum albumin (BSA); 4 μg/mL LDL cholesterol; 200 μg/mL iron-saturated transferrin; 10 μg/ml insulin; 50 μM 2-β-mercaptoethanol, nucleotides (about 20 μM for each of ATP, UTP, GTP, and GTP), and 50 ng/ml thrombopoietin (TPO) in Iscove's Modified Dulbecco's Medium (IMDM). See also U.S. Pat. No. 10,113,147.

In some embodiments, the MKs can be generated/induced from stem cells that are sourced from pluripotent stem cells (PSCs). In some embodiments, the method further includes obtaining PSCs. In some embodiments, the method further includes transducing expression of transcription factors in the PSCs via a vector. In some embodiments, the transcription factors were cloned into a vector backbone. In some embodiments, the vector comprises a lentiviral vector. In some embodiments, the transcription factors include GATA binding protein 1 (GATA1); friend leukemia integration 1 (FLI1); and T-cell acute lymphocytic leukemia protein 1 (TAL1). See also Moreau T, et al. “Large-scale production of megakaryocytes from human pluripotent stem cells by chemically defined forward programming.” Nat Commun. 2016;7:11208. In some embodiments, the method further includes maintaining the transduced PSCs in PSC medium for about 2 days. In some embodiments, the PSC medium includes fibroblast growth factor (FGF2) and Activin-A. In some embodiments, the method further includes maintaining the transduced PSCs in MK medium. In some embodiments, the MK medium includes TPO and SCF for at least 5 days after the maintenance in the PSC medium step.

In some embodiments, the method 100 further includes separating a percentage of the grown MKs and isolating the mitochondria included in the MKs in step 180. In some embodiments, the percentage is 75% and the mitochondria isolated from the percentage is packed into the remaining 25% MKs prior to isolation of the mitochondria from the latter, which thereby increases the copy number of each mitochondria within the 25% MKs. In some embodiments, the percentage is 80%. In some embodiments, the percentage is 70%. In some embodiments, the percentage is from 70% to 80%. In some embodiments, the percentage is 65%. In some embodiments, the percentage is from 65% to 80%. In some embodiments, the method further includes incubation of the MKs with mitochondrial transcription factor A (TFAM) to include mitochondria to increase their copy number.

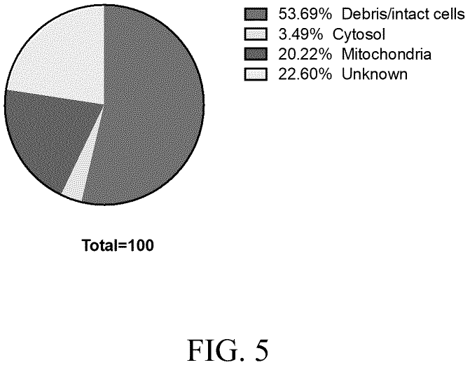

In some embodiments, the method 100 further includes isolating the mitochondria from MKs grown in the bioreactor in step 180. In some embodiments, the isolating step 180 includes incubating the MKs for at least 15 minutes at 4° C. in 500 μL of isolation buffer. In some embodiments, the isolation buffer includes 2 mM HEPES, 10 mM KCl, 1.5 mM MgCl₂, 1 mM EDTA, 1 mM EGTA, 1 mM dithiothreitol (DTT). In some embodiments, the isolation buffer has a pH of 7.5. In some embodiments, the isolating step 180 further includes lysing the MKs. In some embodiments, the lysing step includes using a plastic potter/pestler with 20 strokes and a brief centrifuge spin between each stroke. In some embodiments, the centrifuge spin includes a first spin at 1,000 g for 10mins in 4° C. to gather the supernatant and discard the pellet, the pellet of the first spin including debris and intact cells. In some embodiments, the centrifuge spin includes a second spin at 13,000 g for 15 mins in 4° C. to retrieve the pellet, the pellet of the second spin including the mitochondria. As shown in FIG. 5, the yield for mitochondria improved to 20.22%, a significant improvement to previous methods.

In some embodiments, the lysing step includes treating the MK with extraction buffer. In some embodiments, the lysing step further includes transferring the treated MKs into a dissociating device to homogenize the treated MKs. In some embodiments, the lysing step further includes filtering the homogenized MKs and labeling the mitochondria with magnetic microbeads, the labeled mitochondria still remaining in the homogenate. In some embodiments, the microbeads include Anti-TOM22 microbeads. In some embodiments, the Anti-TOM22 microbeads are human or mouse. In some embodiments, the lysing step further includes applying a magnetic field about a column placed in a separator device and running the labeled mitochondria down the column, wherein the magnetically labeled mitochondria are retained in the column. In some embodiments, the lysing step further includes removal of the column from the separator device and eluting the mitochondria from the column.

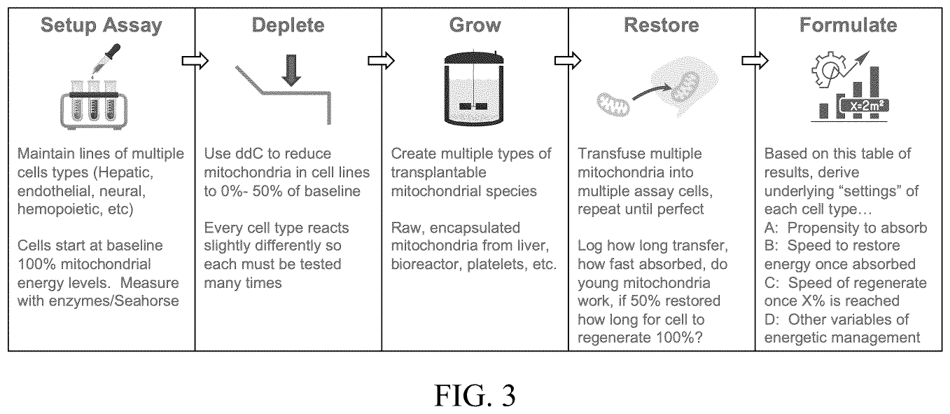

In some embodiments, as shown in FIG. 3, the method includes maintaining lines of multiple cell types. These cell type include, but are not limited to, hepatic cells, endothelial cells, neural cells, hemopoietic cells, or any combination thereof. The cell types included in the method start at a baseline 100% mitochondrial energy level as measured by oxygen consumption rate (OCR) under the Seahorse Assay. In some embodiments, the method further includes reducing mitochondria in cell lines from about 0% to about 50% baseline using 2′,3′ dideoxycytidine (ddC), an agent that depletes mtDNA, which results in severe reduction of mitochondrial activity (and hence a reduction in cellular oxidative respiration). In some embodiments, a health of the cell types is determined after the mitochondria reducing step. In some embodiments, the determining of the cell type health is performed at least one or more times (i.e., cycles) for each of the cell type(s). In some embodiments, the determining of the cell type heath is performed at least a plurality of cycles for each of the cell type(s). In some embodiments, the method further includes growing multiple types of transplantable mitochondrial species. The mitochondrial species can be derived from any source and from any step of the method disclosed herein. In some embodiments, the method further includes restoring cellular energetics by transfusing a plurality of mitochondria from the growing step of the multiple types of transplantable mitochondrial species into multiple assay cells. In some embodiments, the restoring step further includes determining the duration of time required for the mitochondria to transfuse into their target assay cells; and the duration of time required for cellular energetics to reach normal baseline. In some other embodiments, the method further includes deriving characteristics for each cell type, the characteristics including, but not limited to, propensity of the cell type to absorb the transfused mitochondria. Speed of restoration for cellular respiration (e.g., depletion of mtDNA using ddC prior to transfusion of the obtained mitochondria); speed of regeneration of cellular energetics once a threshold has been reached; and other variables of energetic management. In some embodiments, the threshold is 25%, 30%, 35%, 40%, 45%, 50%, 55%, or any integer between 25% and 55%.

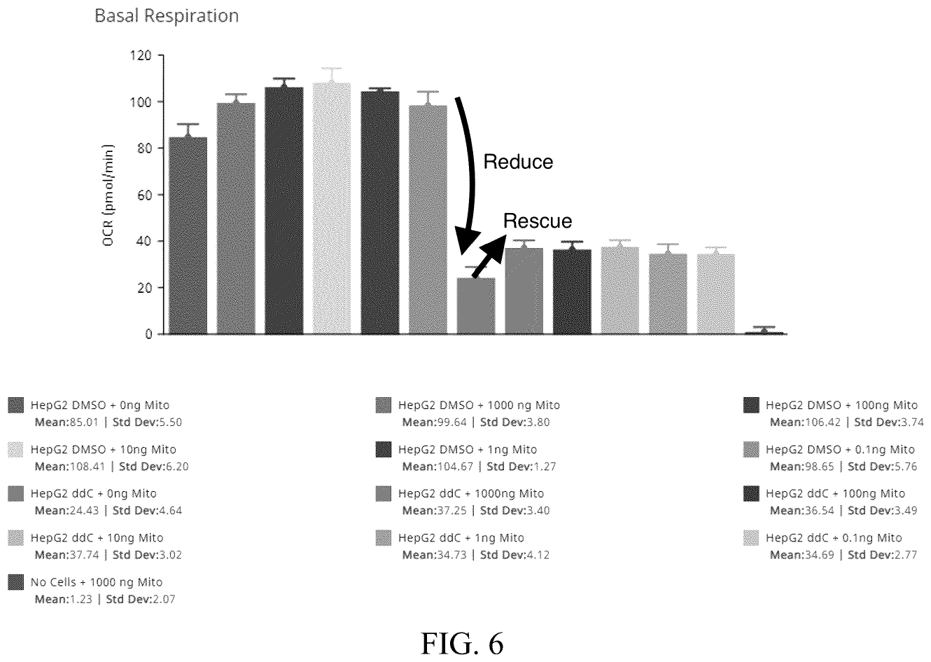

In the non-limiting example shown in FIG. 6, the basal respiration in HepG2 cells (an immortalized human liver cell line), as determined by measurement cellular respiration via OCR (pmol/min), shows a severe reduction of OCR from ˜85% to ˜24.4% incubating ddC in the HepG2 cell culture. From an OCR of about 24.4%±4.6%, administration of 0.1 ng mitos restored the OCR to about 36.7%±2.8%; 10 ng mitos restored OCR to about 37.7%±3.0%; 100 ng mitos restored the OCR to 36.6%±3.5%; 1000 ng of mitos restored OCR to about 37.3%±3.4%. Accordingly, administration of at least 0.1 ng of mitos showed a significant increase of cellular respiration towards basal respiration (as measured by OCR) of HepG2 cells that exhibited significantly diminished cellular respiration following ddC treatment.

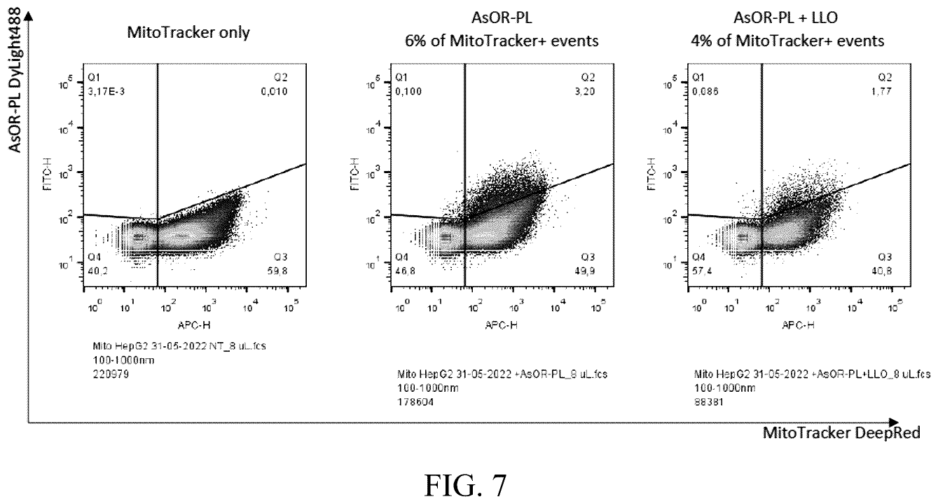

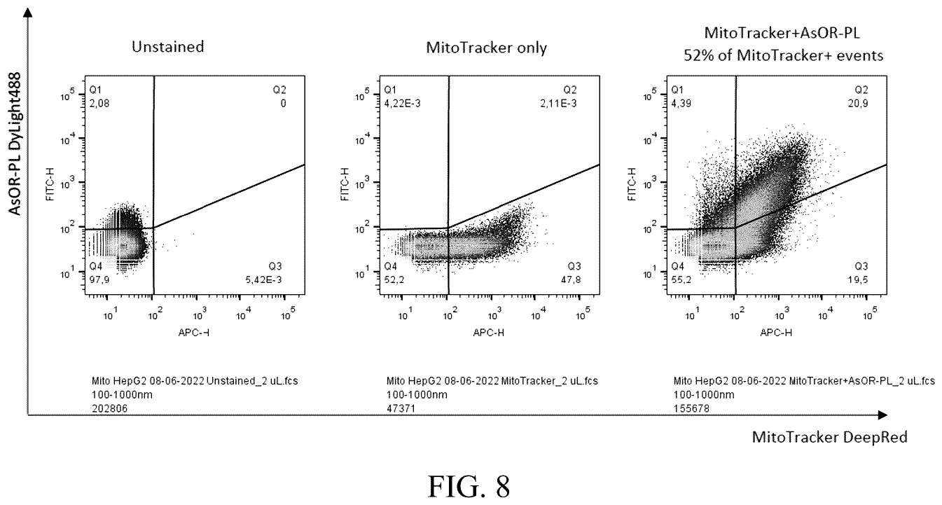

In some embodiments, the method 100 further includes applying a coating to the mitochondria in step 190. In some embodiments, the mitochondria include mitochondria extracted from the MKs. In some embodiments, the coating includes asialoorosomucoid (AsOR). In some embodiments, the coating further includes a poly-L-lysine added to the AsOR. As shown in FIG. 8, the mitos Asialoglycoprotein receptors (AsGRs) function to internalize AsGs with endosomes of cells. Because AsOR can be coated onto cells and AsOR is a ligand for AsGRs, the cellular material or organelles coated with an AsOR-based coating can be targeted for uptake into endosomal compartments of cells. Therefore, mitochondria (sourced from another cell) that is coated by an AsOR-based coating may be internalize into another cell and be delivered to the endosome of that cell. To avoid being targeted for the degradation pathway that leads to the lysosome (e.g., the endosome transfers its contents to the lysosome), the coating further includes listeriolysin O (LLO). LLO is an endosomlytic peptide and the addition thereof in the coating that includes AsOR and PL creates a porin-like channel in the endosome that causes the endosome to rupture from the resultant osmotic pressure, thereby releasing the coated mitochondria into the cell. As shown in FIGS. 7 and 8, the AsOR-PL+LLO and AsOR-PL coating readily associates with mitos as determined by the flow cytometry assay.

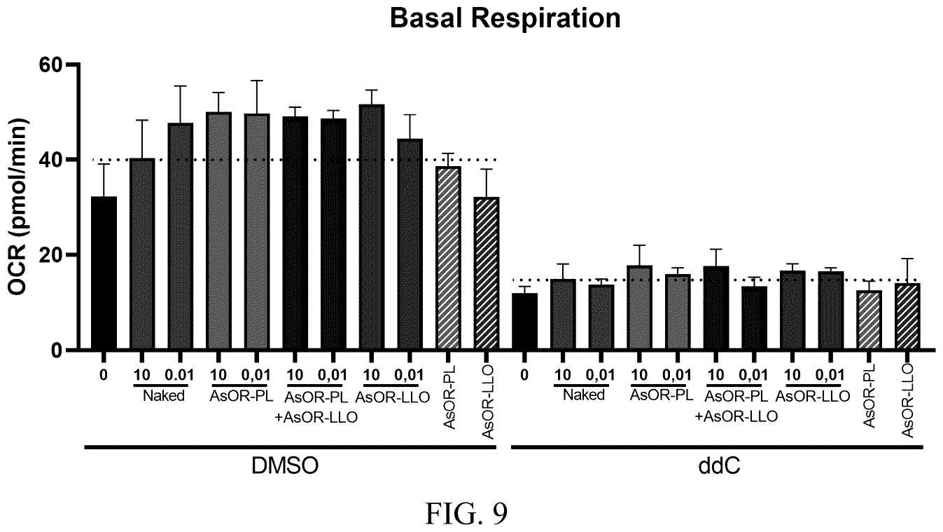

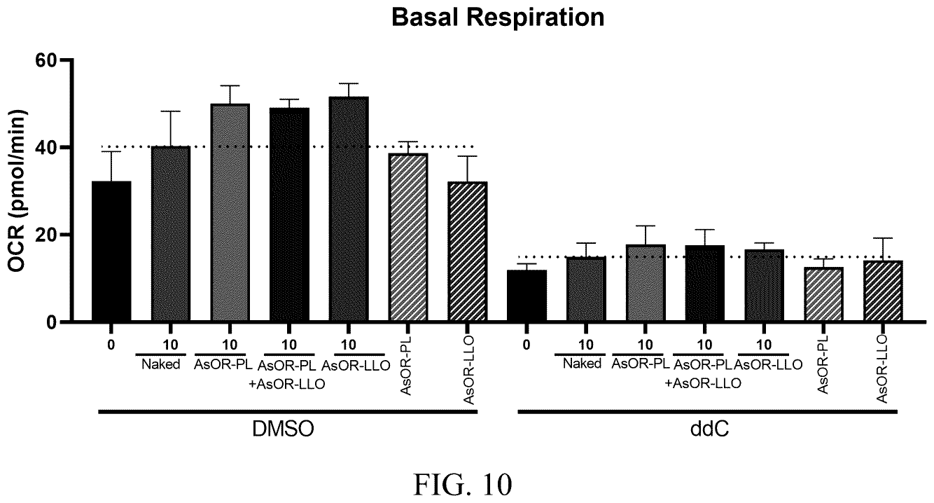

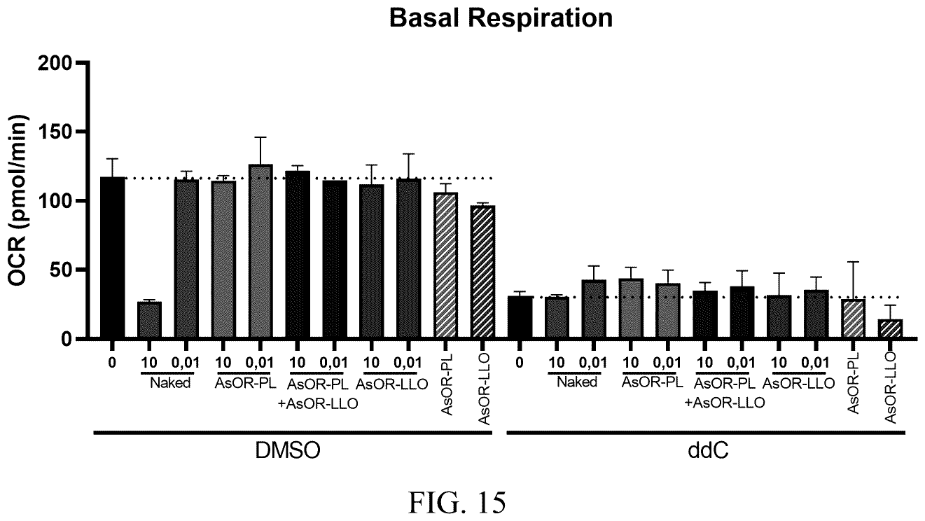

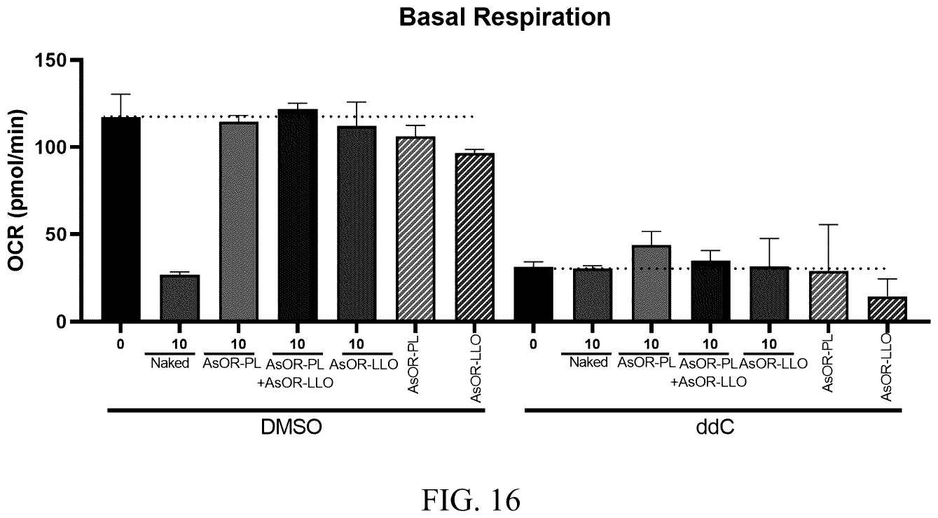

As shown in a non-limiting example in FIGS. 9 and 10, the basal respiration of cells 24 hours of incorporating a particular concentration of mitos (coated or uncoated) displayed more robust basal respiration compared to controls in dimethyl sulfoxide (DMSO) treated cells, particularly those mitos who received the coating: AsOR-PL, AsOR-PL and LLO, or AsOR-LLO. The importance of mitos incorporation is underscored by another group of HepG2 cells incubated in medium containing ddC. Still, the results of FIGS. 9 and 10 show a trend towards rescue of the energy-deficient cells when coated mitochondria of 0.01 pg and 10 pg mitos per cell was administered to the culture. The example of FIG. 7 has HepG2 cells cultivated for a week in normal medium with either DMSO or 10 μM ddC. Mitochondria were extracted from the HepG2 cells. 64 μg of mitochondria were either left untreated, coated with AsOR-PL only, coated with AsOR-PL+AsOR-LLO, or coated with AsOR-LLO only. Mitos (10 pg/cell or 0.01 pg/cell) were added to the cells in medium at +1% serum (to limit cell growth). Cells were incubated with dilutions of AsOR-PL/AsOR-LLO corresponding to the condition 10 pg/cell. Cells were incubated overnight at 37C or for 72 hrs before cellular respiration was assessed. At 72 hours, as shown in FIGS. 15 and 16, the cellular energetics for the mitos of the DMSO-treated HepG2 appear to have dropped back to the baseline. However, naked mitochondria, of which 10 pg/cell was administered to the cell, appear to be severely reduced with regards to the basal respiration of HepG2 cells, which further underscore how naked mitochondria may be deleterious to its target compared to coated cells at similar concentrations. As for the mtDNA depleted ddC-treated HepG2 cells, there appears to be a trend that indicates mito administration as being favorable for increasing the basal respiration rate of those cells.

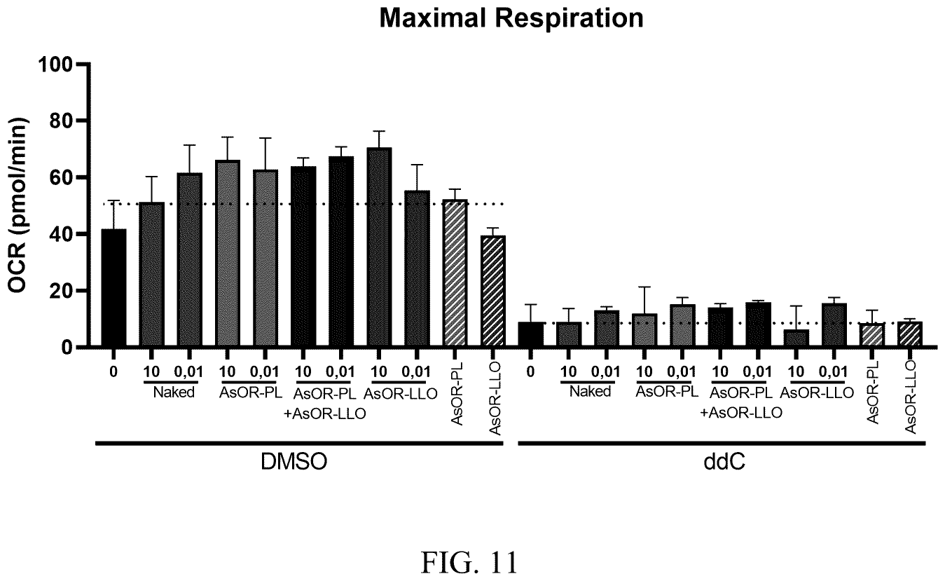

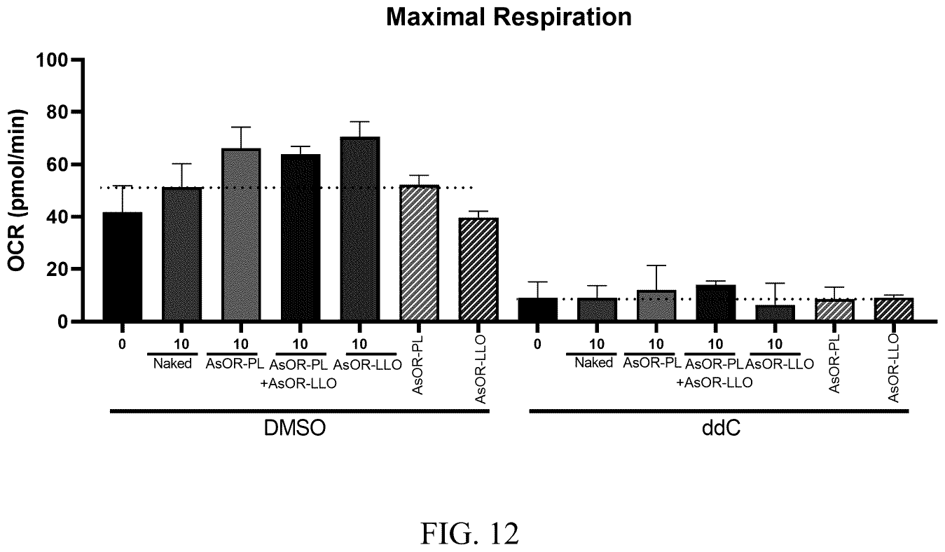

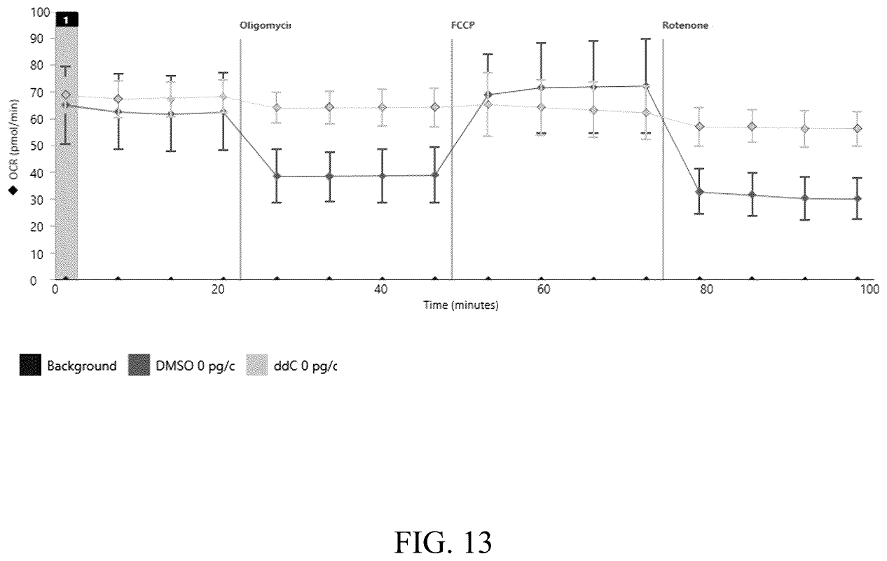

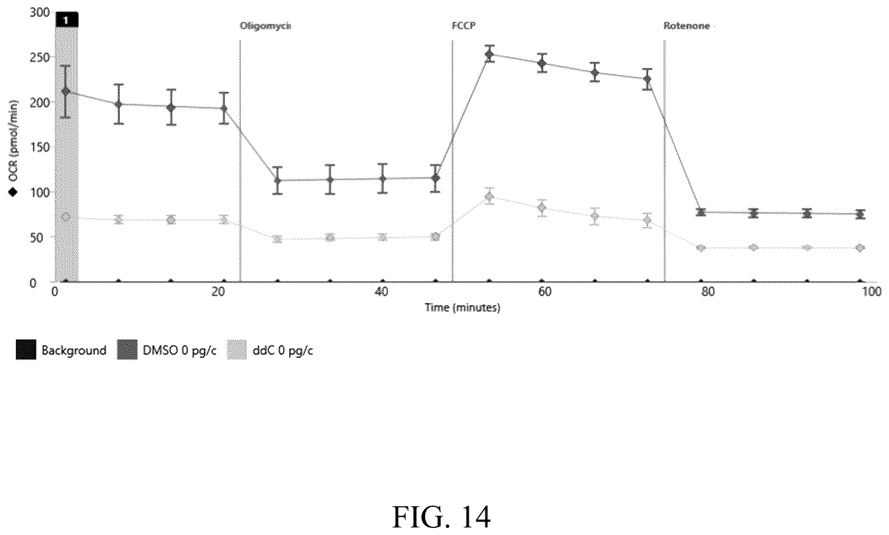

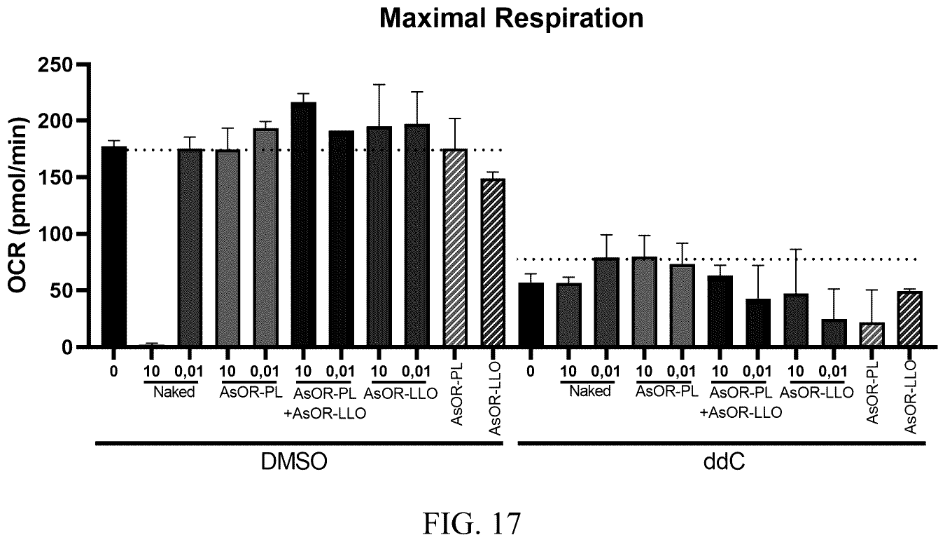

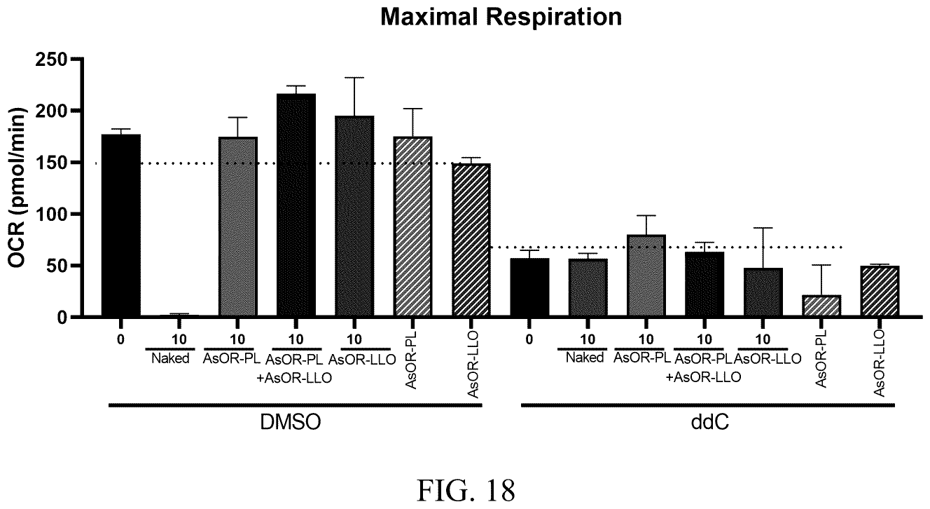

In regards of maximal respiration at 24 hours shown in FIGS. 11 and 12, coated mitos, particularly those having AsOR-PL-PL, exhibited significantly superior OCR compared to the controls. There does not appear to be dose-dependent response to the amount of coated mitos were administered to the HepG2 cells, administration of 0.01 pg of mitos per cell display similar results to the administration of 10 pg of mitos per cell. In terms of rescue of mtDNA depletion brought forth by ddC, lower concentrations (0.01 pg/cell) of coated mitos (AsOR-PL, AsOR-PL+AsOR-LLO, AsOR-LLO) display are more consistent rescue of OCR compared to the administration of 10 pg/cell. FIGS. 13 and 14 show how mtDNA-depleted ddC-treated HepG2 cells (without mitos) exhibited a severe reduction in OCR for all phases tested by the Seahorse Assay: basal respiration (energetic demands of the cell under baseline conditions), ATP production (ATP produced by the mitochondria of the HepG2 cells that contributes to meeting the energetic needs of the cell), proton leak (provides a sign of possible mitochondrial damage), maximal respiration (adding uncoupler FCCP mimics physiological energy demand by stimulating the respiratory chain to operate at max capacity), spare capacity (HepG2 cells capability to respond to demand as an indicator of cell fitness or flexibility, and non-mitochondrial oxygen consumption (persistence of oxygen consumption from a subset of cellular enzymes that continue to consume oxygen follow addition of rotenone and antimycin A). As shown in FIGS. 17 and 18, after 72 hours, the naked mitochondria administered at 10 pg/cell appears to have severely diminished the maximal respiration OCR.

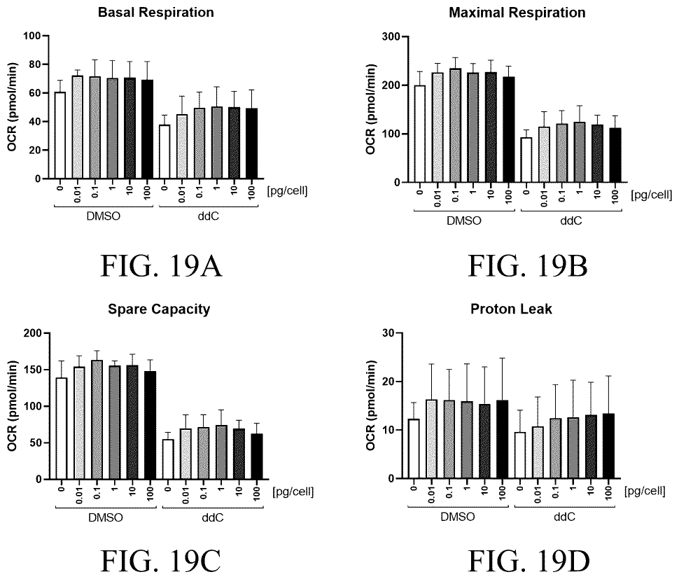

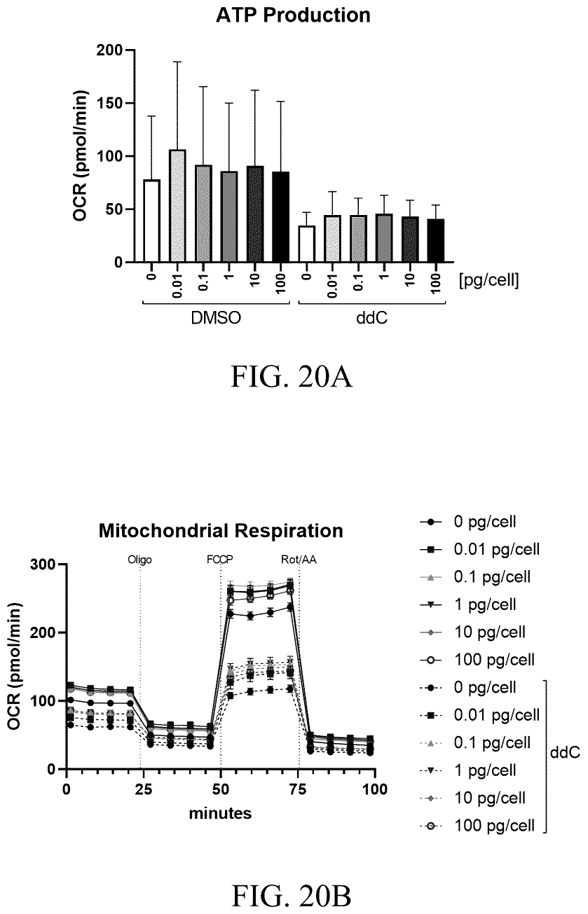

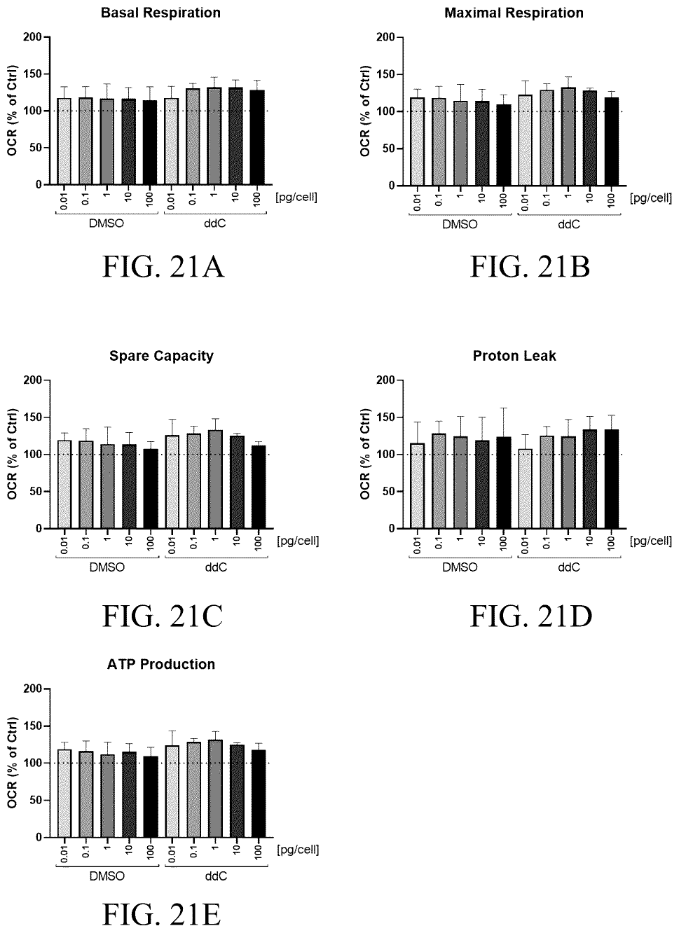

FIG. 19 show the effect of naked mitochondria administration at varying concentrations on ddC-treated retinal pigment epithelium cells (RPECs). Here, the naked mitochondria are uncoated. FIGS. 20A and 20B show ATP production and the overall cellular respiration respectively in ddC-treated RPECs that are administered with naked mitochondria. FIG. 21 shows a compilation of fold change data of ddC-treated RPECs that are treated with naked mitochondria at varying concentrations.

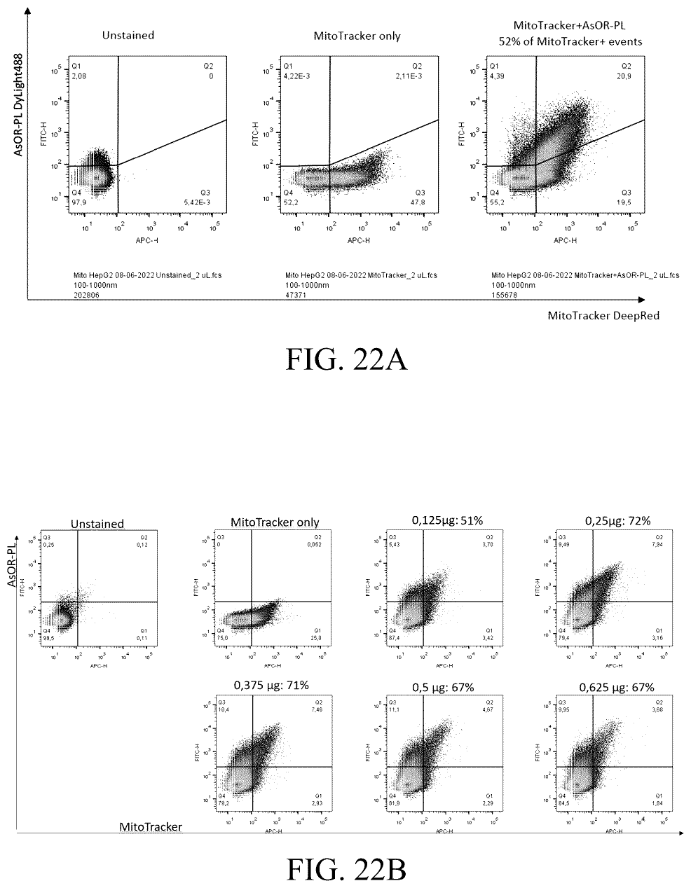

As shown in FIGS. 22A and 22B, the coating can be optimized. In one non-limiting example, various quantities of AsOR-PL on fresh HepG2 mitochondria were tested to determine whether the percentage of coated mitochondria can be increased. Here, mitochondria were extracted from HepG2 cells. These fresh mitos were stained with mitotracker. Mitotracker dyes are fluorescent compounds that are cell-permeable and mitochondrion-selective. These dyes either bind to thiol-reactive chloromethyl groups in the mitochondrial membrane, or bind to free thiol groups of cysteine residues belonging to mitochondrial proteins. As shown in FIG. 22A, the AsOR-PL coating is readily applied on the mitochondria, which represented 52% of the mitotracker events shown in the flow cytometry assay. Typically, conventional methods display yields of about 50% coating of mitochondria. To increase the yield of coated mitochondria, doubling the amount (1 μg of AsOR-PL for 128 μg of mitochondria), a significantly large number of coated mitochondria (about 70-72%) was obtained as shown in FIG. 22B. The typical yield of 51% is shown as 0.125 μg. The increased yields of 72%, 71%, 67%, and 67% are shown for 0.25 μg, 0.375 μg, 0.5 μg, and 0.625 μg respectively.

In some embodiments, the method further includes administration of a therapeutic amount of the bioreactor-grown mitochondria into a subject systemically (enteric or parenteral). In some embodiments, the bioreactor-grown mitochondria is administered to the subject intra-vitreally, intravenously, or intra-arterially. In some embodiments, the bioreactor-grown mitochondria includes a coating. In some embodiments, the coating includes AsOR-PL. In some embodiments, the coating further includes LLO.

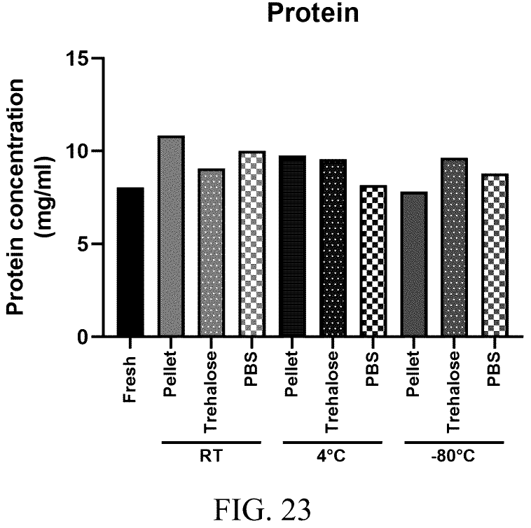

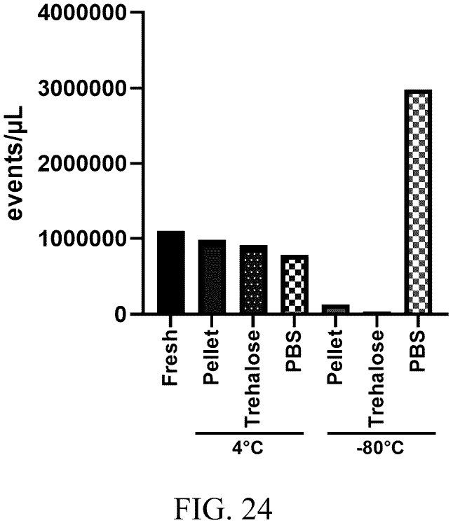

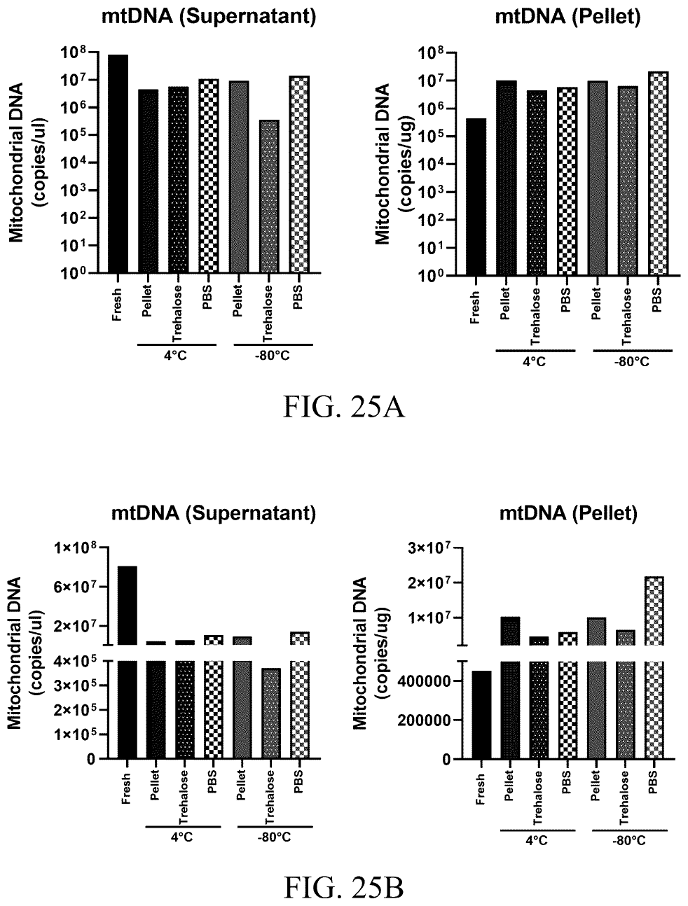

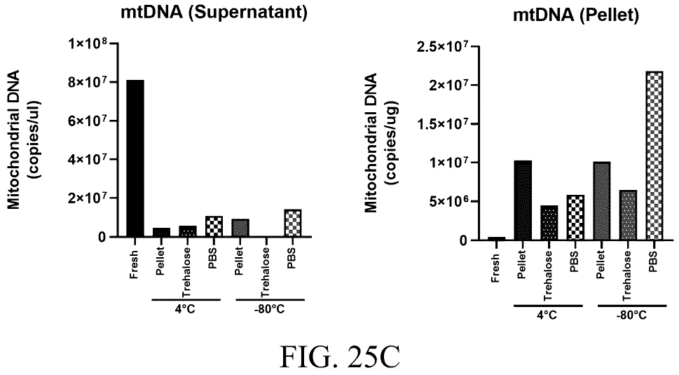

In some embodiments, the method further includes storage of the mitochondria, results of which from non-limiting examples are shown in FIGS. 23-28. Mitochondria were stored at 10 mg/ml (100μg/tube). The conditions were: stored as a pellet; stored in PBS, or stored in trehalose buffer (300 mM trehalose, 10 mM HEPES-KOH PH 7.7, 10 mM KCl, 0.1% BSA, 1 mM EDTA, 1 mM EGTA). Tubes were store at Room Temperature (RT), 4° C. and −80° C. (snap-freezed prior to storage). After 1 week, mitochondria were extracted from a fresh liver for a control. Samples were all tested for: protein concentration (BCA); count of mitochondria (flow cytometry); mtDNA in the supernatant and the cells (qPCR)-for the pellet, they were first resuspended in PBS (same volume than the PBS condition), then centrifuged to harvest the free mitochondrial DNA in the supernatant; ATP production assay (bioluminescence); and Electron Flow Assay (Seahorse). As shown in FIG. 24, at 4° C., protein concentrations were similar between all conditions to fresh mitochondria. At −80° C., the pellet was hard to resuspend. For FIG. 25A, in the supernatant, the trehalose buffer appears to prevent the release of mtDNA during the freezing process at −80° C. In the pellet, the fresh cells also had a lower amount of mtDNA, but still between the storage conditions the mtDNA was quite similar to the fresh mitochondria. For FIGS. 25B and 25C, the mtDNA becomes more concentrated in the pellet following rounds of centrifugation. In FIG. 26, the ATP levels in mitochondria, depending on their storage conditions, were determined. As shown, the luminescence signal that measured the ATP level showed at least a 2× increase in mitochondria stored in some type of solution at room temperature. In some embodiments, the solution comprises phosphate buffered saline (PBS). In some embodiments, the solution comprises trehalose. While dry pellet seemed to have kept well at 4° C., the dry pellet appears not to be a proper storage solution compared to PBS and trehalose at −80° C.

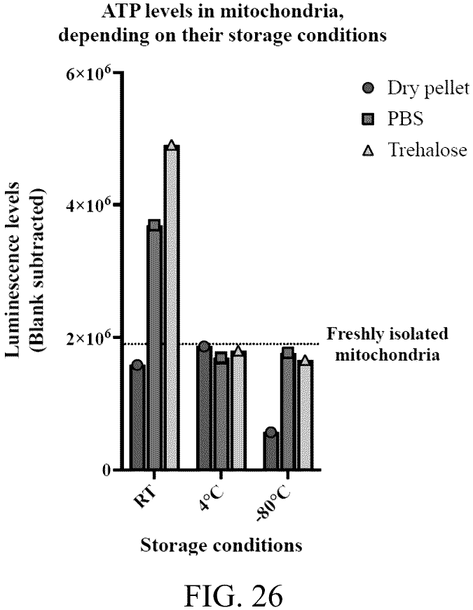

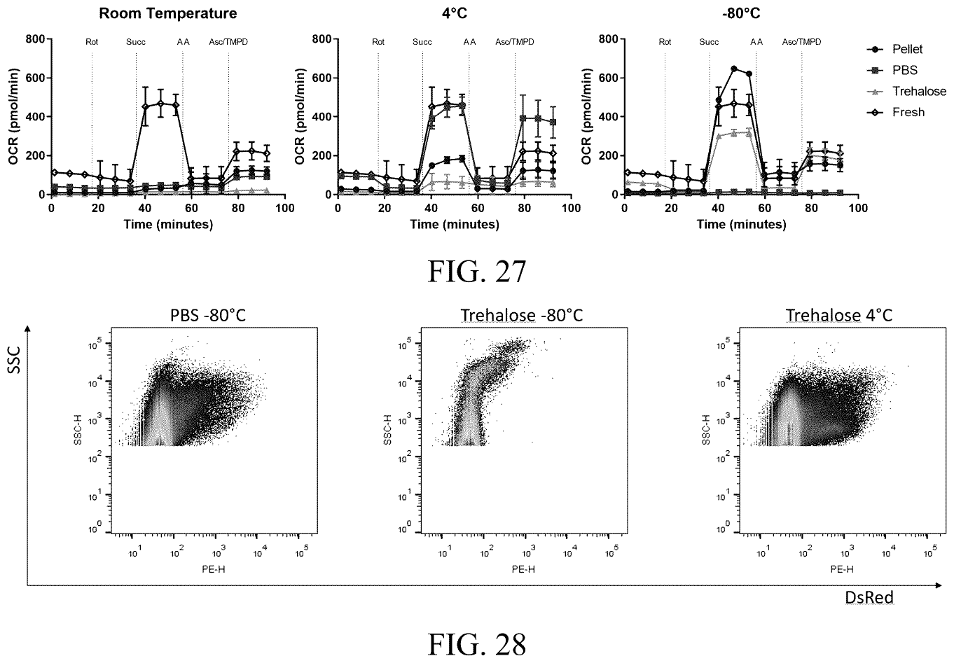

As shown in FIG. 27, the Seahorse assay reveals, physiologically, the PBS and trehalose solutions may not be proper for room temperature storage. However, at cooler temperatures, PBS appears to be advantageous to trehalose at 4° C., whereas trehalose appears to have the advantage over PBS at −80° C. For long-term storage, trehalose appears to be a better option. From the flow cytometry data of FIG. 28, PBS appears to be the preferred option for storage at 4° C. and trehalose appears to be the preferred option for long-term freezing at −80° C.

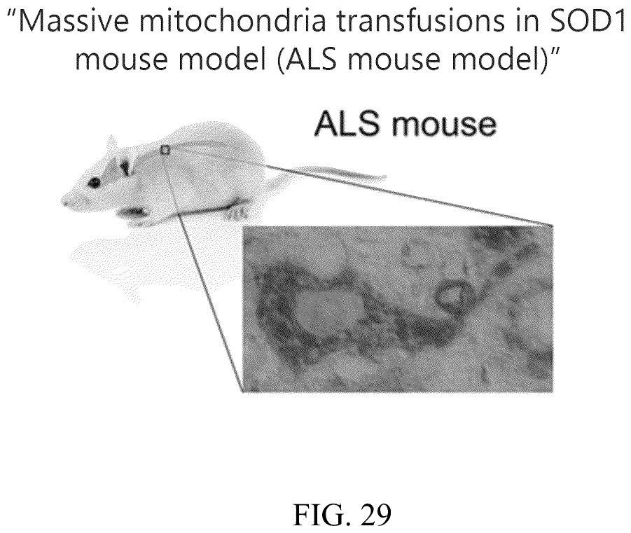

FIG. 29 is a non-limiting example of treating an animal model of amyotrophic lateral sclerosis (ALS) with mitochondria infusions. As used herein, the term “amyotrophic lateral sclerosis” (ALS) refers to a progressive neurodegenerative disease that primarily affects motor neurons in the brain and spinal cord, leading to muscle weakness and eventually paralysis. ALS is characterized by the degeneration and eventual death of both upper motor neurons (UMNs) in the motor cortex of the brain, and lower motor neurons (LMNs) in the brainstem and spinal cord. The specific etiology of ALS is unknown, but it is believed to be caused by a combination of genetic and environmental factors. ALS also encapsulates its clinical manifestations, which typically include muscle weakness, atrophy, fasciculations (muscle twitches), spasticity, and difficulties in speech, swallowing, and respiration. Symptoms generally progress from the site of onset to involve most voluntary muscles. The animal model of ALS includes an SOD1 transgenic mouse (SOD1 mouse), which is a mouse model of ALS. In the SOD1 mouse, a mutated version of the human superoxide dismutase 1 (SOD1) is inserted into the mouse genome and overexpressed.

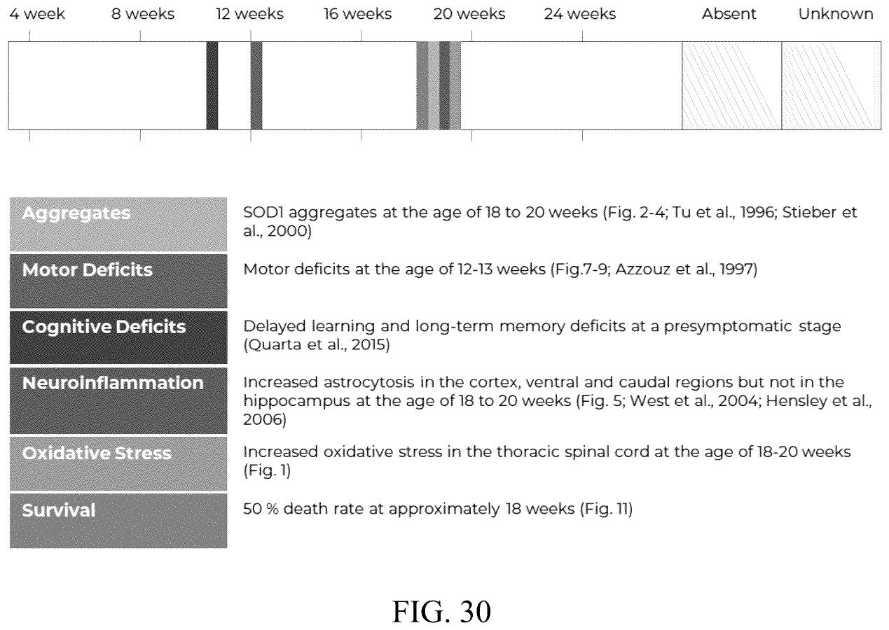

FIG. 30 illustrates several phenotypes from the overexpression of SOD1 in the SOD1 mouse, which include the development of a progressive neurodegenerative disease that closely mimics human ALS, both in terms of its pathological and clinical characteristics. Some of these characteristics include presence of SOD1 aggregates, selective degeneration of motor neurons in the brain and spinal cord, muscle weakness and atrophy, neuroinflammation, oxidative stress, cognitive defects, and a progressive decline in motor function leading to paralysis and eventually death, often due to respiratory failure. These symptoms typically start to manifest when the mice are a few months old and progress rapidly, reflecting the aggressive nature of human ALS. As shown, the survival rate of SOD1 is 50% at approximately 18 weeks.

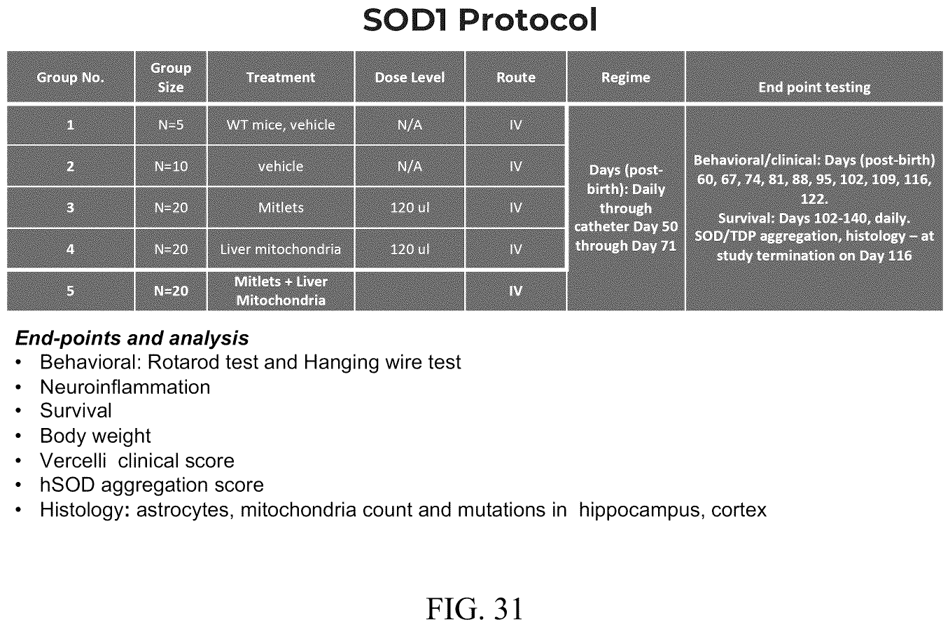

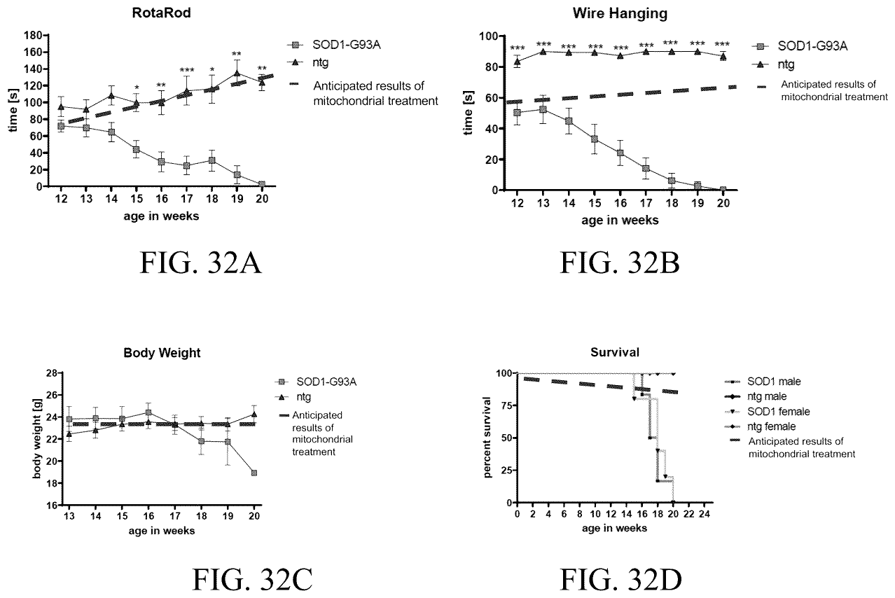

FIG. 31 illustrates a study design to assess the efficacy of mitochondria infusions in the SOD1 mouse model of ALS. To ensure a sufficient amount of mitlets are available, the mitlets are obtained from any of the methods disclosed herein. The SOD1 mice are segregated into five treatment groups. As shown, the Group 1 serves as a negative control group that includes WT mice that receive vehicle; Group 2 serves as a positive control group that includes SOD1 mice that receive vehicle infusions; Group 3 includes SOD1 mice that will receive 120 uL infusions of mitlets; Group 4 includes SOD1 mice that will receive 120 uL infusions of naked mitochondria sourced from mice liver; and Group 5 includes SOD1 mice that will receive mitlets and naked mitochondria sourced from mice liver. These groups will undergo various endpoint testing that includes behavioral/clinical, survival, and SOD/TDP aggregation assessments. The behavioral/clinical assessments include, but are not limited to, rotarod and hanging wire tests, neuroinflammation assays, body weight, hSOD aggregation score, and histological assays. The histological assays include those directed to astrocytes, hippocampus, and the cortex. A non-limiting example includes examining a section of hippocampus to assay mitochondria count and mutations therein. The behavioral tests can be scored using a Vercelli scoring system as a reference standard. Vercelli A, et al. “Human mesenchymal stem cell transplantation extends survival, improves motor performance and decreases neuroinflammation in mouse model of amyotrophic lateral sclerosis.” Neurobiol Dis. 2008;31 (3): 395−405. In the Vercelli scoring system, SOD1 mice were evaluated for signs of motor deficit based on the following point-scoring: 4 points if normal (no sign of motor dysfunction); 3 points if hind limb tremors are evident when suspended by the tail; 2 points if gait abnormalities are present; 1 point for dragging of at least one hind limb; 0 points for inability to right itself within 30 s. Onset of motor deficits is defined retrospectively as the earliest time when the mice showed symptoms (score<4) for ≥2 consecutive weeks. The results are shown in FIG. 32.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise. While several examples have been described in detail, it is to be understood that the disclosed examples may be modified. Therefore, the foregoing description is to be considered non-limiting.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

Treatments may be derived from the methods disclosed herein, treatments that potentially reverse the photoaging process on the face and hands; improve endpoints for neurodegenerative indications (e.g., Alzheimer's, Parkinson's, ALS), as mitochondrial transfusions have regenerated mitochondrial energy in the hippocampus of aged mice; potentially regenerating the retina, one of the most energy-intensive parts of the body in AMD and glaucoma indications; aid the immune system in battling against sepsis and infectious diseases, such as Covid-19, and include potentially reversing immune system senescence; potential anti-aging therapy, affecting strength, cognition, and vitality.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

The described embodiments and examples of the present disclosure are intended to be illustrative rather than restrictive, and are not intended to represent every embodiment or example of the present disclosure, and thus, are not to be limited in scope by the specific embodiments and examples described herein. While the fundamental novel features of the disclosure as applied to various specific embodiments thereof have been shown, described, and pointed out, it will also be understood that various omissions, substitutions, and changes in the details of the methods that are disclosed, may become apparent and may be made by those skilled in the art without departing from the spirit of the disclosure. For example, it is expressly intended that all combinations of those method steps that perform substantially the same function in substantially the same way to achieve the same results are within the scope of the disclosure. Moreover, it should be recognized that method steps shown and/or described in connection with any disclosed form or embodiment of the disclosure may be incorporated in any other disclosed or described or suggested form or embodiment as a general matter of design choice. Further, any of the steps disclosed herein may be repeated. Further, various modifications and variations can be made without departing from the spirit or scope of the disclosure as set forth in the following claims both literally and in equivalents recognized in law.

What is claimed is:

1. A method of growing isolated mitochondria, the method comprising:
selecting a source comprising mitochondria and stem cells;
extracting the stem cells and the mitochondria from the source and segregating the stem cells and the mitochondria into first and second pools respectively;
incubating/transferring the extracted mitochondria into the extracted stem cells to produce packed stem cells;
expanding the packed stem cells in a bioreactor;
adjusting conditions of an environment of the bioreactor to favor growth of the mitochondria of the packed stem cells;
converting the packed stem cells into megakaryocytes;
isolating the mitochondria from the megakaryocytes; and
applying a coating to the mitochondria following isolation thereof from the megakaryocytes.

2. The method of claim 1, wherein the source is placenta tissue, bone marrow, or adipose tissue comprising the mitochondria and the stem cells.

3. The method of claim 1, wherein the coating comprises asialoorosomucoid (AsOR), a poly-L-lysine, or listeriolysin O (LLO).

4. The method of claim 1, further comprising storing the coated mitochondria.

5. The method of claim 4, wherein the storing comprises suspending the mitochondria in a cryoprotectant.

6. The method of claim 5, wherein the cryoprotectant comprises trehalose or phosphate buffered saline (PBS).

7. The method of claim 1, wherein the coating is applied at a ratio of 1 μg of the coating for 128 μg of the bioreactor-grown mitochondria, or at a ratio that is at least double of 1 μg of the coating for 128 μg of the bioreactor-grown mitochondria.

8. The method of claim 1, further comprising administering a therapeutic amount of the coated mitochondria to a subject.

9. The method of claim 1, wherein the source is peripheral blood comprising platelet-derived extracellular vesicles (PEVs), and wherein the PEVs comprise the mitochondria.

10. The method of claim 9, further comprising:
obtaining blood from one or more donors;
adding an anticoagulant and a buffer to the blood to form a mix;
separating the mix into supernatant and platelet rich plasma (PRP);
collecting the PRP;
stimulating the collected PRP, thereby expelling extracellular vesicles from platelets in the PRP; and
collecting the extracellular vesicles as the PEVs.

11. The method of claim 10, wherein the collected PRP is stimulated with immune complexes in presence of Ca²⁺.

12. The method of claim 11, wherein the immune complexes comprise heat-aggregated IgG.

13. The method of claim 12, wherein concentration of the heat-aggregated IgG is 0.1 mg/mL to 2.5 mg/mL, and wherein concentration of the Ca²⁺ is 1 mM to 25 mM.

14. The method of claim 10, wherein the anticoagulant is anticoagulant citrate dextrose (ACD).

15. The method of claim 10, wherein the buffer is Tyrode's buffer at pH 6 to pH 7.

16. A method of growing isolated mitochondria, the method comprising:
selecting a source comprising mitochondria and stem cells;
extracting the stem cells and the mitochondria from the source and segregating the stem cells and the mitochondria into first and second pools respectively;
transferring the extracted mitochondria into the extracted stem cells to produce packed stem cells;
expanding the packed stem cells in a bioreactor;
adjusting conditions of an environment of the bioreactor to favor growth of the mitochondria of the packed stem cells;
converting the packed stem cells into megakaryocytes; and
isolating the mitochondria from the megakaryocytes.

17. The method of claim 16, further comprising applying a coating to the mitochondria following isolation thereof from the megakaryocytes.

18. The method of claim 16, wherein the source is placenta tissue, bone marrow, or adipose tissue, comprising the mitochondria and the stem cells.

19. The method of claim 17, wherein the coating comprises asialoorosomucoid (AsOR), a poly-L-lysine, or listeriolysin O (LLO).

20. The method of claim 17, further comprising storing the coated mitochondria.

21. The method of claim 20, wherein the storing comprises suspending the mitochondria in a cryoprotectant.

22. The method of claim 21, wherein the cryoprotectant comprises trehalose or phosphate buffered saline (PBS).
